(19) United States
(12) Patent Application Publication
(10) Pub. No.: US 2026/0026757 A1
(43) Pub. Date: Jan. 29, 2026

(54) BLOOD PRESSURE MEASUREMENT FOR SYNCOPE EPISODES

(71) Applicant: Medtronic, Inc., Minneapolis, MN (US)

(72) Inventors: Catherine R. Condie, Shoreview, MN (US); Taycia L. Brandon, Otsego, MN (US); Georgina G. Nunez, Irvine, CA (US); Anna J. Malin, Paradise Valley, AZ (US); Ana C. Natera, Somerville, MA (US)

(21) Appl. No.: 19/277,865

(22) Filed: Jul. 23, 2025

Related U.S. Application Data
(60) Provisional application No. 63/675,828, filed on Jul. 26, 2024.

(51) Int. Cl. A61B 5/00; A61B 5/0205; A61B 5/021; A61B 5/11

(52) U.S. Cl. CPC A61B 5/7282; A61B 5/0205; A61B 5/1117; A61B 5/686; A61B 5/742; A61B 5/021

(57) ABSTRACT

Example devices, systems, and techniques are described for determining a current blood pressure for a potential syncope episode. An example system includes processing circuitry configured to determine an occurrence of a potential syncope episode. To determine the occurrence of the potential syncope episode, the processing circuitry is configured to determine at least one of a) entering of patient input on an external device indicative of one or more patient symptoms of syncope, b) changes in a sensed neurological signal, or c) occurrence of a sensed sound of emesis. The processing circuitry is configured to determine, based on the determination of the occurrence of the potential syncope episode, a current blood pressure. The processing circuitry is configured to output an indication of the potential syncope episode and an indication of the current blood pressure.

### RELATED APPLICATIONS

This application claims the benefit of U.S. Provisional Application Ser. No. 63/675,828, filed Jul. 26, 2024 the entire contents of each of which are incorporated herein by reference.

### TECHNICAL FIELD

The disclosure relates generally to devices, systems, and techniques, and more particularly to devices, systems, and techniques to capture blood pressure information.

### BACKGROUND

Implantable medical devices (IMDs) and external, e.g., wearable, medical devices, including implantable pacemakers and implantable cardioverter-defibrillators (ICDs) and insertable cardiac monitors (ICMs) without therapies (e.g., Medtronic LINQ™ or LINQ II™) record cardiac electrogram (EGM) or electrocardiogram (ECG) signals for sensing cardiac events, e.g., P-waves and R-waves. Medical devices detect episodes of bradycardia, tachycardia and/or fibrillation from the sensed cardiac events, and some medical devices respond to the episodes as needed with pacing therapy or high-voltage anti-tachyarrhythmia shocks, e.g., cardioversion or defibrillation shocks. These and other medical devices may include, or be part of a system that includes, sensors that generate other physiological-based signals, such as signals that vary based on patient movement or activity, cardiovascular pressure, blood oxygen saturation, edema, or thoracic impedance.

### SUMMARY

In general, this disclosure is directed to techniques for automatically capturing blood pressure measurements based on the occurrence of syncopal events, possible syncopal events, and/or other triggering events, and providing the blood pressure measurements for analysis by a clinician to assist the clinician in determining and/or understanding a root cause of syncopal events of a patient. More particularly, this disclosure contemplates a device or system that monitors a person for syncopal and/or possible syncopal events and captures blood pressure information associated with such events. In some examples, the device may include an insertable cardiac monitor or other implantable medical device. In some examples, the device may include a wearable device, such as a smart watch or fitness monitor, or the like.

In one example, a system includes one or more memories; and processing circuitry communicatively coupled to the one or more memories, the processing circuitry configured to: determine an occurrence of a potential syncope episode, wherein to determine the occurrence of the potential syncope episode the processing circuitry is configured to determine at least one of a) entering of patient input on an external device indicative of one or more patient symptoms of syncope, b) changes in a sensed neurological signal, or c) occurrence of a sensed sound of emesis; determine, based on the determination of the occurrence of the potential syncope episode, a current blood pressure; and output an indication of the potential syncope episode and an indication of the current blood pressure.

In other examples, a method includes determining, by processing circuitry, an occurrence of a potential syncope episode, wherein to determine the occurrence of the potential syncope episode the processing circuitry is configured to determine at least one of a) entering of patient input on an external device indicative of one or more patient symptoms of syncope, b) changes in a sensed neurological signal, or c) occurrence of a sensed sound of emesis; determining, by the processing circuitry and based on the determination of the occurrence of the potential syncope episode, a current blood pressure; and outputting, by the processing circuitry, an indication of the potential syncope episode and an indication of the current blood pressure.

In other examples, an implantable medical device includes: one or more memories; and processing circuitry communicatively coupled to the one or more memories, the processing circuitry configured to: determine an occurrence of a potential syncope episode, wherein to determine the occurrence of the potential syncope episode the processing circuitry is configured to determine at least one of a) entering of patient input on an external device indicative of one or more patient symptoms of syncope, b) changes in a sensed neurological signal, or c) occurrence of a sensed sound of emesis; determine, based on the determination of the occurrence of the potential syncope episode, a current blood pressure; and output an indication of the potential syncope episode and an indication of the current blood pressure.

This summary is intended to provide an overview of the subject matter described in this disclosure. It is not intended to provide an exclusive or exhaustive explanation of the apparatus and methods described in detail within the accompanying drawings and description below. The details of one or more aspects of the disclosure are set forth in the accompanying drawings and the description below.

### BRIEF DESCRIPTION OF DRAWINGS

FIG. 1 is a conceptual drawing illustrating an example medical device system in conjunction with a patient.

FIG. 2A is a conceptual drawing illustrating an example configuration of the implantable medical device of FIG. 1.

FIG. 2B is a perspective drawing illustrating another example configuration of the implantable medical device of FIG. 1.

FIG. 3 is a block diagram illustrating an example configuration of the implantable medical device (IMD) of FIG. 1.

FIG. 4 is a functional block diagram illustrating an example configuration of an external device configured to communicate with one or more implantable medical devices.

FIG. 5 is a functional block diagram illustrating an example system that includes remote computing devices, such as a server and one or more other computing devices, that are connected to an implantable medical device and/or external device via a network.

FIG. 6 is a flowchart illustrating example techniques for taking a blood pressure measurement for a potential syncope episode according to one or more aspects of this disclosure.

### DETAILED DESCRIPTION

The occurrence of syncope symptoms in a patient may be related to underlying cardiac or other issues, e.g., neurological, situational (stress-related, illness-related, etc.), or the like. Syncope episodes may be dangerous, in and of themselves. For example, a patient may lose consciousness when driving due to a syncope episode.

When addressing syncope episodes, a clinician may desire to treat the underlying cause of the syncope. Cardiac-related syncope may be caused by a pause, bradycardia, supraventricular tachycardia (SVT), or ventricular tachycardia (VT), for example. Another common cause of syncope is drops in blood pressure due to a vasovagal response, positional changes, or other neurological responses. These may or may not have a cardiac change associated with them. As such, it may be desirable to capture, and provide to a clinician, blood pressure information around (e.g., associated with) syncopal or possible syncopal events. Possible syncopal events may include falls and/or other posture changes which may be indicative of syncope. Blood pressure information may enable a clinician to better determine the underlying cause of the syncope and to tailor treatment(s) to that root cause, thereby improving patient outcomes, patient quality of life, as well as the safety of the patient and those around the patient.

Implantable cardiac monitors (ICMs), such as LINQ™ monitors, may be used to help diagnose the cause of unexplained syncope in patients. ICMs monitors can be used to determine if the underlying cause is cardiac related—i.e., a pause, bradycardia, SVT or VT. Another common cause of syncope is drops in blood pressure due to a vasovagal response, positional changes, or other neurological responses. These may or may not have a cardiac change associated with them. This disclosure relates to techniques intended to provide blood pressure information around syncopal or possible syncopal events (such as falls or other posture changes) to help clinicians and patients determine, treat, or understand the root cause of such events.

In some examples, a device or system may monitor for possible syncopal events and perform a blood pressure measurement based on detecting a possible syncopal event. In some examples, the device or system may perform blood pressure measurements based on a time or an event-based trigger. In some examples, the device or system may determine a treatment/efficacy of a treatment for the unexplained syncope. Because the techniques of this disclosure may be implemented in an insertable cardiac monitor or other IMD, the techniques may be performed while the patient is ambulatory taking part in activities of everyday life. Such a device or system may be useful as clinician cannot be present with the patient during many activities of everyday life. The clinician may miss vital and useful information in evaluating and addressing the patient's state of health when potential syncope episodes occur outside of the doctor's office. Because the time spent in a doctor's office is a relatively small portion of the time in the lives of most patients, determination of causes of syncope episodes and/or effective treatments may be difficult based solely on doctor's office visits. Indeed, syncope episodes may come and go and may not be present when in the doctor's office. For example, syncope episodes may be caused by factors not encounterable in doctor's office, such as environmental factors, etc.

For example, a clinician may desire to determine whether syncope symptoms or a syncope episode is due to a relatively large or drastic change in blood pressure, or due to something else. Therefore, a blood pressure measurement associated with each potential syncope episode may be desirable. However, it is not feasible for a person to always wear a blood pressure cuff while participating in the activities of daily life. Additionally, determining blood pressure by an implantable medical device may be a relatively high-power task, which may drain a battery relatively quickly if the IMD determines the blood pressure continually. As such, it may be desirable to have a system that includes an IMD that may detect a syncopal or pre-syncopal event and, based on the detection of the syncopal or pre-syncopal event, determine a blood pressure of the patient.

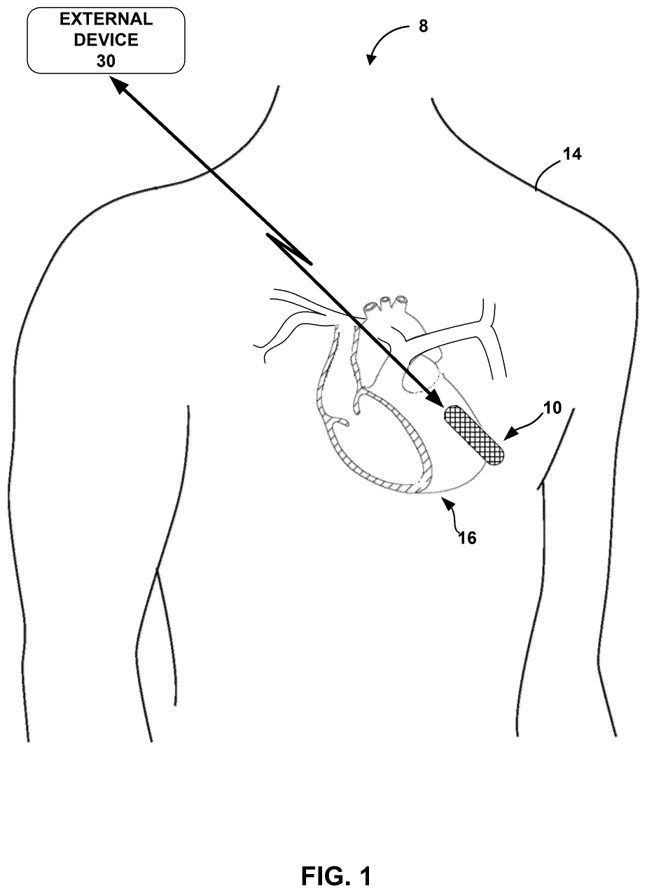

FIG. 1 is a conceptual diagram illustrating an example medical device system 8 in conjunction with a patient 14. Medical device system 8 is an example of a medical device system configured to implement the techniques described herein for automatically capturing blood pressure information. In the illustrated example, medical device system 8 includes an IMD 10 and an external device 30.

In some examples, system 8 may include IMD 10 which may trigger and/or perform a blood pressure measurement based on the detection of a possible syncopal event. In other words, the blood pressure measurement may be trigged by the detection of the possible syncopal event. IMD 10 may detect the possible syncopal event through any of, or any combination of: 1) determining a fall or other posture change based on accelerometer signal(s), such as from a 3-axis accelerometer; 2) determining changes in the heart rate or heart rhythm; 3) receiving input from a patient indicating syncope symptoms using an external device or phone communicating with the IMD 10; 4) determining changes in respiration as sensed by the IMD; 5) various sensor data from a wearable (accelerometer, smart watch, . . . etc.)—through Bluetooth communication with IMD 10 or external device 30; 6) determining changes in neurological signals (e.g., sympathetic, parasympathetic, brain waves, . . . etc.); 7) determining dizziness of patient 14 (e.g., changes in walking pattern) based on accelerometer signal(s); and/or 8) determining of the sound or action of emesis [vomiting], which often occurs with a syncope episode, based on one or more signals from a microphone or an accelerometer of IMD 10.

IMD 10 may be an insertable cardiac monitor (ICM) capable of sensing and recording ECG signals from a position outside of heart 16. Further, IMD 10 is capable of implementing one or more techniques for automatically capturing blood pressure information in accordance with one or more aspects of the present disclosure. In some examples, IMD 10 includes or is coupled to one or more sensors that generate one or more other physiological signals, such as neurological signals, and/or signals that vary based on patient motion and/or posture (e.g., accelerometer circuitry), blood flow, or respiration. In some examples, IMD 10 may also include a microphone configured to capture sounds made by patient 14, such as sounds of emesis or vomiting, which may be indicative of a potential syncope episode. IMD 10 may be implanted in patient, subcutaneously or submuscularly, such as the pectoral location illustrated in FIG. 1. In some examples, IMD 10 may take the form of a LINQ™ ICM, or LINQ II™ ICM available from Medtronic, Inc., of Minneapolis, Minnesota.

External device 30 may wirelessly communicate with IMD 10, e.g., to program the functionality of the ICM, and to retrieve recorded physiological signals and/or patient parameter values, or other data derived from such signals from the ICM In some examples, external device 30 may include a user interface configured for a patient to enter patient input indicative of one or more patient symptoms of syncope. Both IMD 10 and external device 30 include processing circuitry, and the processing circuitry of either or both devices may perform the techniques described herein for capturing blood pressure information, as discussed in further detail below.

Although not illustrated in the example of FIG. 1, a system configured to implement the techniques of this disclosure may include one or more implanted or external medical devices in addition to or instead of IMD 10. For example, a medical device system may include a pressure sensing IMD, vascular ICD, extravascular ICD, cardiac pacemaker, neurologic signal sensing device and/or the like. One or more such devices may generate accelerometer signals, heart rate signals, heart rhythm signals, respiration signals, and/or capture a sound of patient 14, and include processing circuitry configured to perform, in whole or in part, the techniques described herein for capturing blood pressure information. In the case where the system includes a plurality of medical devices, the implanted medical device(s) and/or external medical device(s) may communicate with each other and/or an external device 30, and one of (or a combination of) the implanted or external devices may ultimately determine when to capture the blood pressure information.

In some examples, system 8 may consider patient 14 medical history, such as the existence of peripheral artery disease or other diseases states. Such disease states may be used to rule out certain conditions as the cause for a potential syncope episode. System 8 display the blood pressure measurement on episode records and/or fall detection episodes, for example, on a user interface of external device 30. Such data may be displayed to a user, such as a clinician or patient 14, which may facilitate correlation of the potential syncope episode and/or current blood pressure with other symptoms.

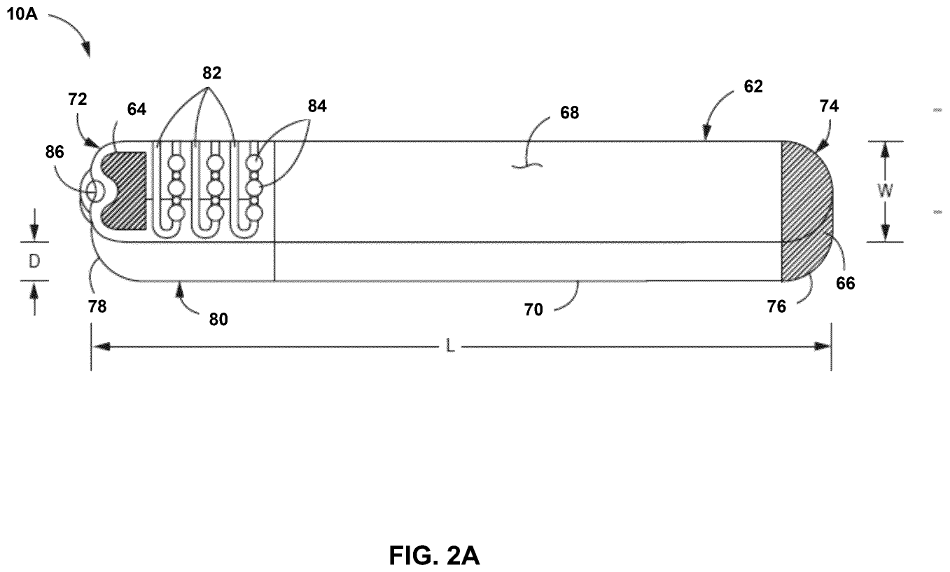

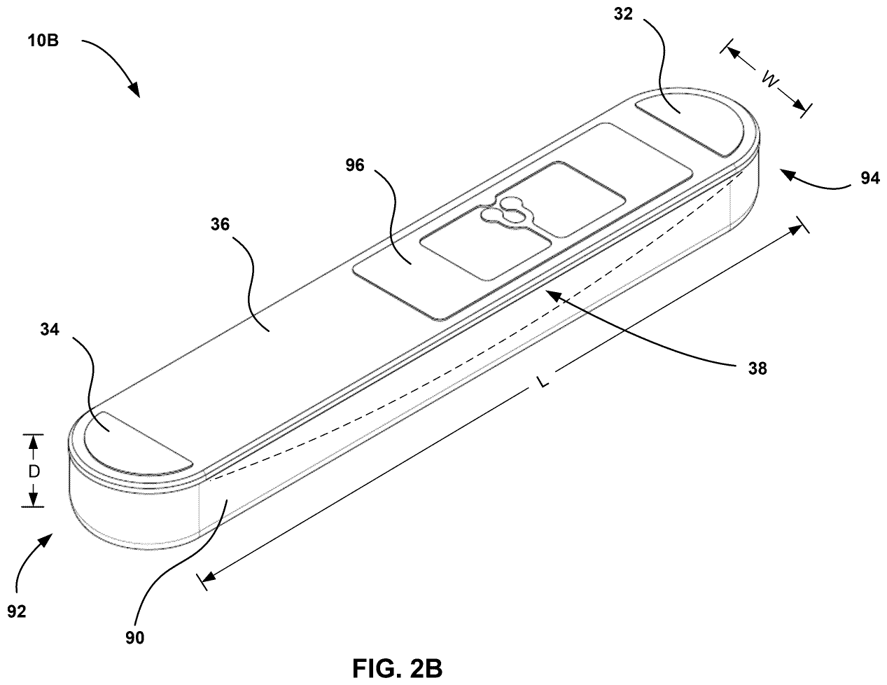

FIG. 2A is a conceptual drawing illustrating an example configuration of IMD 10. In the example shown in FIG. 2A, IMD 10A, which may be an example of IMD 10 of FIG. 1, may be implemented as a monitoring device having housing 62, proximal electrode 64 and distal electrode 66. Housing 62 may further comprise first major surface 68, second major surface 70, proximal end 72, and distal end 74. Housing 62 encloses electronic circuitry located inside the IMD 10A and protects the circuitry contained therein from body fluids. Electrical feedthroughs provide electrical connection of electrodes 64 and 66.

In the example shown in FIG. 2A, IMD 10A is defined by a length L, a width W and thickness or depth D and is in the form of an elongated rectangular prism wherein the length L is much larger than the width W, which in turn is larger than the depth D. In one example, the geometry of the IMD 10A—in particular a width W greater than the depth D—is selected to allow IMD 10A to be inserted under the skin of patient 14 using a minimally invasive procedure and to remain in the desired orientation during insertion. For example, the device shown in FIG. 2A includes radial asymmetries (notably, the rectangular shape) along the longitudinal axis that maintains the device in the proper orientation following insertion. For example, in one example the spacing between proximal electrode 64 and distal electrode 66 may range from 30 millimeters (mm) to 55 mm, 35 mm to 55 mm, and from 40 mm to 55 mm and may be any range or individual spacing from 25 mm to 60 mm. In addition, IMD 10A may have a length L that ranges from 30 mm to about 70 mm. In other examples, the length L may range from 40 mm to 60 mm, 45 mm to 60 mm and may be any length or range of lengths between about 30 mm and about 70 mm. In addition, the width W of major surface 68 may range from 3 mm to 10 mm and may be any single or range of widths between 3 mm and 10 mm. The thickness of depth D of IMD 10A may range from 2 mm to 9 mm. In other examples, the depth D of IMD 10A may range from 2 mm to 5 mm and may be any single or range of depths from 2 mm to 9 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic centimeters.

In the example shown in FIG. 2A, once inserted within patient 14, the first major surface 68 faces outward, toward the skin of patient 14 while the second major surface 70 is located opposite the first major surface 68. In addition, in the example shown in FIG. 2A, proximal end 72 and distal end 74 are rounded to reduce discomfort and irritation to surrounding tissue once inserted under the skin of patient 14.

Proximal electrode 64 and distal electrode 66 are used to sense cardiac signals, e.g., ECG signals, intra-thoracically or extra-thoracically, which may be sub-muscularly or subcutaneously. ECG signals may be stored in a memory of the IMD 10A, and ECG data may be transmitted via integrated antenna 82 to another medical device, which may be another implantable device or an external device, such as external device 30. In some example, electrodes 64 and 66 may additionally or alternatively be used for sensing any bio-potential signal of interest, which may be, for example, an EGM, electroencephalogram (EEG), electromyogram (EMG), or a nerve signal, from any implanted location.

In the example shown in FIG. 2A, proximal electrode 64 is in close proximity to the proximal end 72 and distal electrode 66 is in close proximity to distal end 74. In this example, distal electrode 66 is not limited to a flattened, outward facing surface, but may extend from first major surface 68 around rounded edges 76 and/or end surface 78 and onto the second major surface 70 so that the electrode 66 has a three-dimensional curved configuration. In the example shown in FIG. 2A, proximal electrode 64 is located on first major surface 68 and is substantially flat, outward facing. However, in other examples proximal electrode 64 may utilize the three-dimensional curved configuration of distal electrode 66, providing a three-dimensional proximal electrode (not shown in this example). Similarly, in other examples distal electrode 66 may utilize a substantially flat, outward facing electrode located on first major surface 68 similar to that shown with respect to proximal electrode 64.

The various electrode configurations allow for configurations in which proximal electrode 64 and distal electrode 66 are located on both first major surface 68 and second major surface 70. In other configurations, such as that shown in FIG. 2A, only one of proximal electrode 64 and distal electrode 66 is located on both major surfaces 68 and 70, and in still other configurations both proximal electrode 64 and distal electrode 66 are located on one of the first major surface 68 or the second major surface 70 (i.e., proximal electrode 64 located on first major surface 68 while distal electrode 66 is located on second major surface 70). In another example, IMD 10A may include electrodes on both major surface 68 and 70 at or near the proximal and distal ends of the device, such that a total of four electrodes are included on IMD 10A. Electrodes 64 and 66 may be formed of a plurality of different types of biocompatible conductive material, e.g., stainless steel, titanium, platinum, iridium, or alloys thereof, and may utilize one or more coatings such as titanium nitride or fractal titanium nitride.

In the example shown in FIG. 2A, proximal end 72 includes a header assembly 80 that includes one or more of proximal electrode 64, integrated antenna 82, anti-migration projections 84, and/or suture hole 86. Integrated antenna 82 is located on the same major surface (i.e., first major surface 68) as proximal electrode 64 and is also included as part of header assembly 80. Integrated antenna 82 allows IMD 10A to transmit and/or receive data. In other examples, integrated antenna 82 may be formed on the opposite major surface as proximal electrode 64, or may be incorporated within housing 62 of IMD 10A. In the example shown in FIG. 2A, anti-migration projections 84 are located adjacent to integrated antenna 82 and protrude away from first major surface 68 to prevent longitudinal movement of the device. In the example shown in FIG. 2A, anti-migration projections 84 includes a plurality (e.g., nine) small bumps or protrusions extending away from first major surface 68. As discussed above, in other examples anti-migration projections 84 may be located on the opposite major surface as proximal electrode 64 and/or integrated antenna 82. In addition, in the example shown in FIG. 2A header assembly 80 includes suture hole 86, which provides another means of securing IMD 10A to patient 14 to prevent movement following insert. In the example shown, suture hole 86 is located adjacent to proximal electrode 64. In one example, header assembly 80 is a molded header assembly made from a polymeric or plastic material, which may be integrated or separable from the main portion of IMD 10A.

FIG. 2B is a perspective drawing illustrating another IMD 10B, which may be another example configuration of IMD 10 of FIG. 1. IMD 10B of FIG. 2B may be configured substantially similarly to IMD 10A of FIG. 2A, with differences between them discussed herein.

IMD 10B may include a leadless, subcutaneously-implantable monitoring device, e.g. an ICM. IMD 10B includes housing having a base 90 and an insulative cover 36. Proximal electrode 32 and distal electrode 34 may be formed or placed on an outer surface of cover 36. Various circuitries and components of IMD 10B, e.g., described below with respect to FIG. 2B, may be formed or placed on an inner surface of cover 36, or within base 90. In some examples, a battery or other power source of IMD 10B may be included within base 90. In the illustrated example, antenna 96 is formed or placed on the outer surface of cover 36, but may be formed or placed on the inner surface in some examples. In some examples, insulative cover 36 may be positioned over an open base 90 such that base 90 and cover 36 enclose the circuitries and other components and protect them from fluids such as body fluids.

Circuitries and components may be formed on the inner side of insulative cover 36, such as by using flip-chip technology. Insulative cover 36 may be flipped onto a base 90. When flipped and placed onto base 90, the components of IMD 10B formed on the inner side of insulative cover 36 may be positioned in a gap 38 defined by base 90. Electrodes 32 and 34 and antenna 96 may be electrically connected to circuitry formed on the inner side of insulative cover 36 through one or more vias (not shown) formed through insulative cover 36. Insulative cover 36 may be formed of sapphire (i.e., corundum), glass, parylene, and/or any other suitable insulating material. Base 90 may be formed from titanium or any other suitable material (e.g., a biocompatible material). Electrodes 32 and 34 may be formed from any of stainless steel, titanium, platinum, iridium, or alloys thereof. In addition, electrodes 32 and 34 may be coated with a material such as titanium nitride or fractal titanium nitride, although other suitable materials and coatings for such electrodes may be used.

In the example shown in FIG. 2B, the housing of IMD 10B defines a length L, a width W and thickness or depth D and is in the form of an elongated rectangular prism wherein the length L is much larger than the width W, which in turn is larger than the depth D, similar to IMD 10A of FIG. 2A. For example, the spacing between electrodes 32 and 34 may range from 30 millimeters (mm) to 50 mm, from 35 mm to 45 mm, or be approximately 40 mm. In addition, IMD 10B may have a length L that ranges from 30 mm to about 70 mm. In other examples, the length L may range from 40 mm to 60 mm, 45 mm to 55 mm, or be approximately 45 mm. In addition, the width W may range from 3 mm to 10 mm, such as approximately 8 mm. The thickness of depth D of IMD 10B may range from 2 mm to 9 mm, from 3 to 5 mm, or be approximately 4 mm. IMD 10B may have a volume of three cubic centimeters (cm) or less, or 1.5 cubic cm or less, such as approximately 1.4 cubic cm.

In the example shown in FIG. 2B, once inserted subcutaneously within the patient, outer surface of cover 36 generally faces outward, toward the skin of the patient. In addition, as shown in FIG. 2B, proximal end 94 and distal end 92 are rounded to reduce discomfort and irritation to surrounding tissue once inserted under the skin of the patient. In addition, edges of IMD 10B may be rounded. In some cases, IMD 10B may flip within a patient such that the outer surface of cover 36 may not face outward, toward the skin of the patient. The techniques of this disclosure may be implementable whether the outer surface of cover 36 faces outward, toward the skin of the patient or not. For example, accelerometer signals may be affected by the orientation of IMD 10B. In some examples, IMD 10B may implement a device flip detection feature which may determine that IMD 10B has flipped based on one or more accelerometer signals and may process such accelerometer signals to adjust for the flipping of IMD 10B.

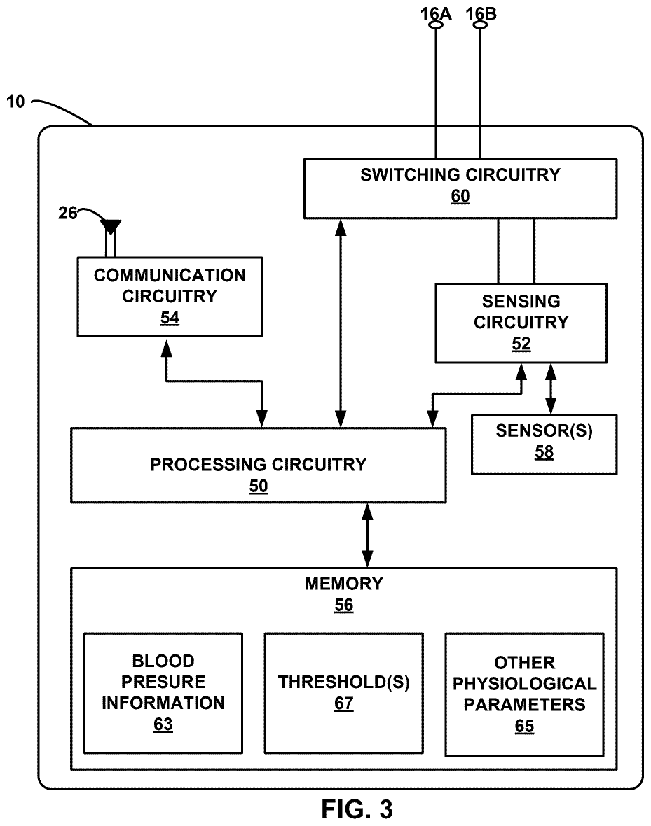

FIG. 3 is a block diagram illustrating an example configuration of IMD 10 of FIG. 1. In some examples, IMD 10 may represent IMD 10A of FIG. 2A or IMD 10B of FIG. 2B. As shown in FIG. 3, IMD 10 includes processing circuitry 50, sensing circuitry 52, communication circuitry 54, memory 56, sensors 58, switching circuitry 60, and electrodes 16A, 16B (hereinafter “electrodes 16”), one or more of which may be disposed on a housing of IMD 10. In some examples, memory 56 includes computer-readable instructions that, when executed by processing circuitry 50, cause IMD 10 and processing circuitry 50 to perform various functions attributed herein to IMD 10 and processing circuitry 50. Memory 56 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media. Memory 56 may also store blood pressure information 63, other physiological parameters 65, and threshold(s) 67. Blood pressure information 63 may include a baseline blood pressure and/or other measures of blood pressure sensed by sensing circuitry 52 and/or sensor(s) 58 as described herein. Other physiological parameters 65 may include signals such as ECG signals, signals indicative of respiration rate, neurological signals. In addition to, or alternatively, physiological parameters 65 may include determined dyspnea scores, determined respiration rates, determined tidal volumes, and/or determined minute ventilations.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16A, 16B via switching circuitry 60 as controlled by processing circuitry 50. Sensing circuitry 52 may monitor signals from electrodes 16A, 16B in order to monitor various physiological parameters of patient 14 of FIG. 1. Processing circuitry 50 may control sensing circuitry to capture blood pressure, for example, via a pressure sensor (e.g., an optical pressure sensor) of sensor(s) 58 or an external, wearable blood pressure monitor. Processing circuitry 50 may control communication circuitry 54 to output indication(s) of determined potential syncope episodes to external device 30. In some examples, processing circuitry 50 may control sensing circuitry 52 and or sensors 58 (which may include an accelerometer, an optical sensor, microphone(s), or other sensors) to sense or determine a change in neurological signals of patient 14, an occurrence of a sound of emesis, a fall of patient 14, a change in a heart rate and/or heart rhythm of patient 14, a change in respiration (e.g., respiration rate, tidal volume, etc.), dizziness of the patient (e.g., based on at least one accelerometer signal), and/or a change in posture of patient 14.

Processing circuitry 50 may process any of such signals (or combination thereof) to determine an occurrence of a potential syncope episode. Processing circuitry 50 may, based on the determination of the potential syncope episode, determine a current blood pressure. For example, processing circuitry 50 may control a blood pressure sensor, such as an optical sensor or a wearable blood pressure sensing device, to determine a current blood pressure. Processing circuitry 50 may generate an indication for output that of the potential syncope episode and an indication of the current blood pressure. In some examples, processing circuitry 50 may also control communication circuitry 54 to output the indications and/or other information, such as physiological parameters 65, for example, to guide a clinician in treatment of patient 14. For example, the clinician may further assess the current blood pressure and/or any other physiological parameters to verify the potential syncope episode, to determine a possible cause for the syncope episode, and/or to determine a treatment for the syncope episode. The techniques of this disclosure may assist a clinician in determining the best course of treatment for patient 14.

Sensing circuitry 52 and/or processing circuitry 50 may be configured to process received signals, such as accelerometer signals, pressure sensor signals, microphone signals, neurological signals (e.g., EEG signals), ECG signals, and/or the like to determine the occurrence of a potential syncope episode and may include filters, peak detectors, envelope calculations, in some examples.

In some examples, IMD 10 includes one or more sensors 58, such as one or more accelerometers, microphones, optical sensors, temperature sensors, pressure sensors, and/or other sensors. In some examples, sensing circuitry 52 may include one or more filters and amplifiers for filtering and amplifying signals received from one or more of electrodes 16A, 16B and/or other sensors 58. In some examples, sensing circuitry 52 and/or processing circuitry 50 may include a rectifier, filter and/or amplifier, a sense amplifier, comparator, and/or analog-to-digital converter. Processing circuitry 50 may determine values of physiological parameters of patient 14 based on signals from sensors 58, which may be used to determine an occurrence of a potential syncope episode in patient 14.

Communication circuitry 54 may include any suitable hardware, firmware, software or any combination thereof for communicating with another device, such as external device 30. Communication circuitry 54 may be configured to communicate using any of a variety of wireless communication schemes, such as Bluetooth® or Bluetooth Low Energy®. Under the control of processing circuitry 50, communication circuitry 54 may receive downlink telemetry from, as well as send uplink telemetry to, external device 30 or another device with the aid of an internal or external antenna, e.g., antenna 26. In some examples, processing circuitry 50 may communicate with a networked computing device via an external device (e.g., external device 30) and a computer network, such as the Medtronic CareLink® Network developed by Medtronic, Inc.

Although described herein in the context of example IMD 10, the techniques for taking a blood pressure measurement disclosed herein may be used with other types of devices. For example, the techniques may be implemented with an extra-cardiac defibrillator coupled to electrodes outside of the cardiovascular system, a transcatheter pacemaker configured for implantation within the heart, such as the Micra™ transcatheter pacing system commercially available from Medtronic, Inc., a neurostimulator, a drug delivery device, a medical device external to patient 14, a wearable device such as a wearable cardioverter defibrillator, a fitness tracker, or other wearable device, a mobile device, such as a mobile phone, a “smart” phone, a laptop, a tablet computer, a personal digital assistant (PDA), or “smart” apparel such as “smart” glasses, a “smart” patch, a “smart” watch, and/or external device 30. In some examples, the techniques described herein may be implemented by a combination of devices. For example, processing circuitry 50 may perform some of the techniques, while processing circuitry of one or more other devices may perform other of the techniques.

In some examples, processing circuitry 50 may trigger a blood pressure measurement based on the detection of a possible syncopal event. Processing circuitry 50 may detect the possible syncopal event through any of, or any combination of the following conditions: 1) determining a fall or other posture change based on accelerometer signal(s), such as from a 3-axis accelerometer; 2) determining changes in the heart rate or heart rhythm; 3) receiving input from a patient indicating syncope symptoms using an external device or phone communicating with the IMD 10; 4) determining changes in respiration as sensed by IMD 10; 5) various sensor data from a wearable (accelerometer, smart watch, . . . etc.)—through Bluetooth communication with IMD 10 or external device 30; 6) determining changes in neurological signals (e.g., sympathetic, parasympathetic, brain waves, . . . etc.); 7) determining dizziness of patient 14 (e.g., changes in walking pattern, staggering) based on accelerometer signal(s); and/or 8) determining of the sound or action of emesis (vomiting), which often occurs with a syncope episode, based on one or more signals from a microphone or an accelerometer of IMD 10. Each of these conditions (1-8) may be indicative of a possible syncope episode. In some examples, IMD 10 may determine a potential syncope episode based on any one of these conditions (1-8) or any combination of any number of these determinations.

For example, processing circuitry 50 may determine a fall of patient 14 based on one or more accelerometer signals, which may be indicative of a potential syncope episode. For example, processing circuitry 50 may employ techniques set forth in U.S. Pat. No. 8,814,811, issued on Aug. 26, 2014, and entitled “FALL DETECTION ALGORITHM UTILIZING A THREE-AXIS ACCELEROMETER,” which is hereby incorporated by reference, to determine a fall of patient 14.

For example, processing circuitry 50 may determine changes in the heart rate or heart rhythm of patient 14. For example, processing circuitry 50 may monitor an ECG sensed by sensing circuitry 52 and when a change in a heart rate in the ECG satisfies a threshold (e.g., is greater than or greater than or equal to the threshold), the change may be indicative of a potential syncope episode. Similarly, processing circuitry 50 may monitor an ECG sensed by sensing circuitry 52 and when a change in a heart rhythm in the ECG satisfies one or more predetermined conditions, the change may be indicative of a potential syncope episode. For example, processing circuitry 50 may compare measure(s) of change(s) in R-R wave variability, morphologies, QRS duration, R-R interval, atrial fibrillation detection, changes in ST segments, asystole detection, etc., to one or more thresholds to determine whether a change in heart rhythm in the ECG satisfies the one or more predetermined conditions.

For example, processing circuitry 50 may receive input from a patient indicative of syncope symptoms. For example, processing circuitry 50 may receive, via communication circuitry 54 and from external device 30, patient input indicative of one or more patient symptoms of syncope. In some examples, the patient input may include the pushing of an icon or virtual button on a touch screen of external device 30. For example, UI 204 may include a touch screen which may display an icon or virtual button which patient 14 may push or touch to indicate to IMD 10 that patient is experiencing symptoms of syncope. A patient experiencing symptoms of syncope may be indicative of a potential syncope episode.

For example, processing circuitry 50 may determine changes in respiration as sensed by IMD 10. For example, processing circuitry 50 may determine a respiration rate, a tidal volume, or the like, based on sensed EGMs, impedance measurements, breathing sounds captured by a microphone, abnormal breathing patterns (such as wheezing, coughing, aspiration, etc.) or the like. Processing circuitry 50 may monitor the respiration signal(s) and when a change in a respiration signal satisfies a threshold (e.g., is greater than or greater than or equal to the threshold), the change may be indicative of a potential syncope episode.

For example, processing circuitry 50 may obtain, via communication circuitry 54, various sensor data from external device 30, which, in some examples, may be a wearable device (e.g., accelerometer, smart watch, . . . etc.). In some examples, such sensor data is similar to other sensor data discussed herein. Processing circuitry 50 may monitor such sensor data for data indicative of a potential syncope episode.

For example, processing circuitry 50 may determine changes in neurological signals (e.g., sympathetic, parasympathetic, brain waves, etc.). For example, processing circuitry 50 may monitor impedance signals, EEGs, or the like of patient 14. Processing circuitry 50 may monitor such signal(s) and when a change in such signal(s) satisfies a threshold (e.g., is greater than or greater than or equal to the threshold), the change may be indicative of a potential syncope episode.

For example, processing circuitry 50 may determine dizziness of patient 14. For example, processing circuitry 50 may monitor one or more accelerometer signals. If processing circuitry 50 determines that the accelerometer signals are indicative of an erratic gate (e.g., stumbling rather than walking), such accelerometer signals may be indicative of a potential syncope episode.

For example, processing circuitry 50 may monitor microphone signal(s) of a microphone of sensor(s) 58, which may capture sounds. Processing circuitry 50 may process such captured sounds, or signals representative thereof, to determine whether captured sounds represent the sound of emesis or vomiting. Emesis or vomiting may be indicative of a potential syncope episode.

In some examples, processing circuitry 50 may trigger and/or perform a blood pressure measurement based on a time or an event-based trigger. For example, processing circuitry 50 may determine a time of day and determine that the time of day is a predetermined time of day. Processing circuitry 50 may, based on the time of day being the predetermined time of day, control sensor(s) 58 to determine a baseline blood pressure. For example, processing circuitry 50 may control a pressure sensor to perform the blood pressure measurement in response to determining the patient is first waking up (which alternatively may be determined by processing circuitry based on accelerometer signals), around meals, at nighttime, at a set time after the patient takes blood pressure medicine, etc. Such a blood pressure measurement may provide a patient-specific individualized baseline blood pressure for which to compare a suspected syncopal event current blood pressure measurement. For example, a blood pressure of 100/60 may be low for one person, but normal for another. As such, a patient-specific baseline blood pressure may be useful in assessing a possible syncope episode, determining a possible cause for the possible syncope episode, determining a recommended treatment for the possible syncope episode, and/or the like. A triggered blood pressure measurement may utilize an optical sensor of sensor(s) 58, or a wearable blood pressure monitor.

Processing circuitry 50 may control communication circuitry 54 to output an indication of the baseline blood pressure. In some examples, processing circuitry 50 may compare the baseline blood pressure to the current blood pressure. In some examples, processing circuitry 50 may determine which information to include in the indication of the potential syncope episode based on the comparison. For example, if the baseline blood pressure is similar or close to (e.g., within a predetermined range of) the current blood pressure, processing circuitry 50 may include less information or different information than if the baseline blood pressure is different or not close to (e.g., outside of the predetermine range of) the current blood pressure. For example, if the baseline blood pressure is not close to the current blood pressure, processing circuitry 50 may include more physiological parameter data and/or more sensor signal data or different physiological parameter data and/or different sensor signal data in the indication of the potential syncope episode. In some examples, processing circuitry 50 may assign a priority of transmission to the indication of the potential syncope episode based on the comparison. For example, if the baseline blood pressure is not close to the current blood pressure, processing circuitry 50 may assign a higher priority to transmission of the indication of the potential syncope episode than if the baseline blood pressure is close to the current blood pressure.

Since vasovagal syncope is often associated with posture changes, in some examples, the system may trigger and/or perform a blood pressure measurement when there is a sudden posture change (e.g., as sensed by the accelerometer of the IMD) to see if there is a significant blood pressure change that may not cause syncope at that particular time, but could be an indicator of pathologic changes in blood pressure indicative of a future possible problem. Such a sudden posture change and associated blood pressure measurement may be similar to a tilt table test, but be captured ambulatorily. This blood pressure measurement may be part of a trend, or a single event “presyncope” event. For example, processing circuitry 50 may determine an occurrence of a triggering event, such as a sudden posture change. Processing circuitry 50 may control a pressure sensor to determine, based on the occurrence of the triggering event, a blood pressure. Processing circuitry 50 may control communication circuitry 54 to output an indication of the blood pressure. In some examples, the triggering event includes at least one of the patient being in a predetermined posture or a rate of change of a posture of the patient satisfying a threshold.

In some examples, processing circuitry 50 may record a patient posture when a blood pressure measurement is made. This may help distinguish between types of syncope episodes, as neurological syncope episodes do not often occur when laying down. For example, processing circuitry 50 may determine, based on the determination of the syncope episode, a patient posture. For example, processing circuitry 50 may use one or more accelerometer signals to determine the patient posture. Processing circuitry 50 may output an indication of the patient posture, for example, along with the indication of the potential syncope episode and the current blood pressure.

In some examples, processing circuitry 50 may control a blood pressure sensor to take a blood pressure measurement based on a particular posture or a posture change, even in the absence of any syncope symptoms. For example, processing circuitry 50 may monitor posture of patient 14 over time. If patient 14 has a potential syncope event, processing circuitry 50 may determine a posture prior to (or at the beginning of) the potential syncope event and may record that posture. Processing circuitry 50 may then use that particular posture as a trigger to control a pressure sensor to take a blood pressure measurement whenever processing circuitry 50 determines patient 14 is in that particular posture.

In some examples, processing circuitry 50 may determine potential causes of the potential syncope episode and/or suggested treatments of the potential syncope episode. In some examples, the indication of the potential syncope episode may include potential causes of the potential syncope episode and/or suggested treatments of the potential syncope episode. For example, the indication of the potential syncope episode may include a suggestion that the clinician titrate blood pressure medication. In some examples, such as where the indication of the potential syncope episode is directed to patient 14, the indication of the potential syncope episode may include instructions to patient 14 to sit down for their own safety. For example, different causes of syncope symptoms may be best treated by different treatments.

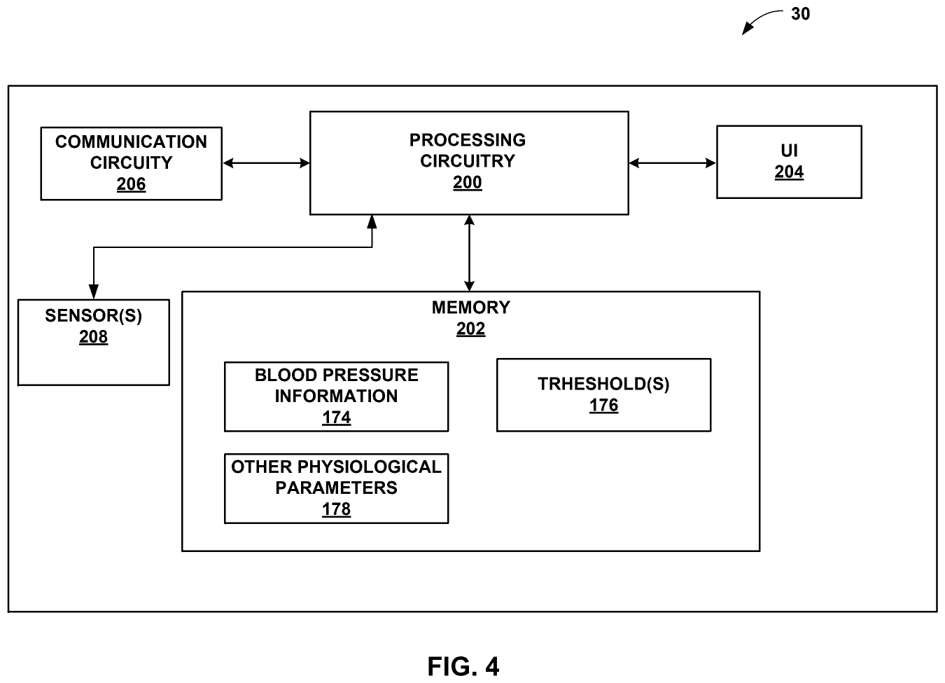

FIG. 4 is a functional block diagram illustrating an example configuration of an external device 30. External device 30 is configured to communicate IMD 10. In some examples, external device 30 is configured to implement the techniques of this disclosure. In some examples, external device 30 may be a smart phone or a wearable device, such as a smart watch or fitness tracker.

In the example of FIG. 4, external device 30 includes processing circuitry 200, memory 202, user interface (UI) 204, communication circuitry 206, and sensors 208. External device 30 may be a dedicated hardware device with dedicated software for the programming and/or interrogation of an IMD 10. Alternatively, external device 30 may be an off-the-shelf computing device, e.g., a smart phone running a mobile application that enables external device 30 to program and/or interrogate IMD 10. In some examples where external device 30 is a smart phone, external device 30 may include a mobile application to facilitate interaction with IMD 10. In some examples, external device 30 may be a wearable device, such as a smart watch or fitness tracker.

In some examples, a user of external device 30 may be clinician, physician, heath care giver, patient, family member of the patient or friend of the patient. In some examples, a user uses external device 30 to select or program any of the values for operational parameters of IMD 10. In some examples, a user uses external device 30 to receive data collected by IMD 10, such as indications of a potential syncope episode, indications of a current blood pressure, other physiological parameters, sensed signals, and/or other operational and performance data of IMD 10. In some examples, the user may also receive alerts provided by IMD 10 that indicate that an acute cardiac event, e.g., ventricular tachyarrhythmia, is predicted. In some examples, IMD 10 may transmit an alert and the user may also receive the alert. The user may interact with external device 30 via UI 204, which may include a display to present a graphical user interface to a user, and a keypad or another mechanism (such as a touch sensitive screen) for receiving input from a user. External device 30 may communicate wirelessly with IMD 10 using communication circuitry 206, which may be configured for RF communication with communication circuitry 54 of IMD 10. Sensors 208 may include accelerometer circuitry, microphone(s), and/or a blood pressure sensor. In some examples, sensors 208, including any accelerometer circuitry, microphone(s), and/or blood pressure sensor, may operate similarly to sensor(s) 58. For examples, sensors 208 may be used to generate signals indicative of a potential syncope episode and/or to obtain a current blood pressure.

In the case where external device 30 is a patient device, in some examples, UI 204 may include a touch screen and may present an icon or virtual button on the touch screen for a user to provide input. In some examples, the icon or virtual button may be used to provide input that patient 14 is experiencing syncope symptoms. In some examples, UI 204 may provide patient 14 which other ways in which to input information indicative of patient 14 experiencing syncope symptoms. In response to such patient input, processing circuitry 200 may control communication circuitry 206 to send an indication to IMD 10 that patient 14 is experiencing syncope symptoms. In some examples, UI 204 may also provide patient 14 with an ability to enter other patient symptom data, which external device 30 may send to TNID 10.

Processing circuitry 200 may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, processing circuitry 200 may include multiple components, such as any combination of one or more microprocessors, one or more DSPs, one or more ASICs, or one or more FPGAs, as well as other discrete or integrated logic circuitry, and/or analog circuitry. Processing circuitry 200 may be communicatively coupled elements of external device 30 such as memory 202, and sensor(s) 208.

Memory 202 may store blood pressure information 174, threshold(s) 176, and/or other physiological parameters 178 which may be similar to, or examples of, blood pressure information 63, threshold(s) 67, and other physiological parameters 65 of FIG. 3. Memory 202 may also store program instructions, which may include one or more program modules, which are executable by processing circuitry 200. When executed by processing circuitry 200, such program instructions may cause processing circuitry 200 and external device 30 to provide the functionality ascribed to them herein. The program instructions may be embodied in software, firmware and/or RAMware. Memory 202 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

In some examples, processing circuitry 200 of external device 30 may be configured to provide some or all of the functionality ascribed to processing circuitry 50 of IMD 10 herein. For example, processing circuitry 200 may obtain sensed signals from sensor(s) 208 and/or from IMD 10 and may determine, based on such signals, an occurrence of a potential syncope episode. Processing circuitry 200 may determine a current blood pressure. For example, processing circuitry 200 may control IMD 10 to determine a current blood pressure, processing signals from IMD 10 to determine a current blood pressure, or control another sensor, such as an optical sensor of sensors(s) 208 or an external pressure sensor device, to determine the current blood pressure of patient 14. In some examples, processing circuitry 200 may detect a possible syncopal event through any of, or any combination of the following conditions: 1) determining a fall or other posture change based on accelerometer signal(s), such as from a 3-axis accelerometer; 2) determining changes in the heart rate or heart rhythm; 3) receiving input from a patient indicating syncope symptoms using an external device or phone communicating with the IMD 10; 4) determining changes in respiration as sensed by IMD 10; 5) various sensor data from a wearable (accelerometer, smart watch, . . . etc.)—through Bluetooth communication with IMD 10 or external device 30; 6) determining changes in neurological signals (e.g., sympathetic, parasympathetic, brain waves, . . . etc.); 7) determining dizziness of patient 14 (e.g., changes in walking pattern, staggering) based on accelerometer signal(s); and/or 8) determining of the sound or action of emesis (vomiting), which often occurs with a syncope episode, based on one or more signals from a microphone or an accelerometer of IMD 10.

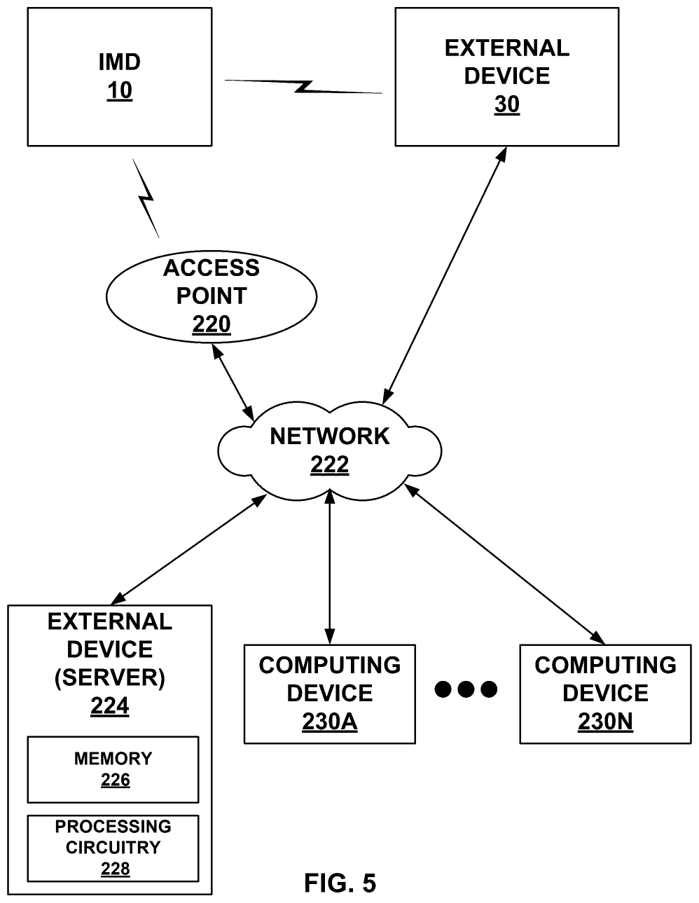

FIG. 5 is a functional block diagram illustrating an example system that includes external computing devices, such as a server 224 and one or more other computing devices 230A-230N, that are coupled to IMD 10 and external device 30 via a network 222. In this example, IMD 10 may use its communication circuitry 54 to, e.g., at different times and/or in different locations or settings, communicate with external device 30 via a first wireless connection, and to communication with an access point 220 via a second wireless connection. In the example of FIG. 9, access point 220, external device 30, server 224, and computing devices 230A-230N are interconnected, and able to communicate with each other, through network 222.

Access point 220 may comprise a device that connects to network 222 via any of a variety of connections, such as telephone dial-up, digital subscriber line (DSL), or cable modem connections. In other examples, access point 220 may be coupled to network 222 through different forms of connections, including wired or wireless connections. In some examples, access point 220 may be co-located with patient 14. Access point 220 may interrogate IMD 10, e.g., periodically or in response to a command from patient 14 or network 222, to retrieve physiological signals, indications of occurrences of potential syncope episodes, blood pressure measurements, alerts of acute cardiac events, and/or other operational or patient data from IMD 10. Access point 220 may provide the retrieved data to server 224 via network 222.

In some cases, server 224 may be configured to provide a secure storage site for data that has been collected from IMD 10 and/or external device 30. In some cases, server 224 may assemble data in web pages or other documents for viewing by trained professionals, such as clinicians, via computing devices 230A-230N. The illustrated system of FIG. 9 may be implemented, in some aspects, with general network technology and functionality similar to that provided by the Medtronic CareLink® Network developed by Medtronic plc, of Dublin, Ireland.

In some examples, one or more of access point 220, server 224, or computing devices 230 may be configured to perform, e.g., may include processing circuitry configured to perform, some or all of the techniques described herein, e.g., with respect to processing circuitry 50 of IMD 10 and processing circuitry 200 of external device 30, relating to determining an occurrence of a potential syncope episode. In the example of FIG. 9, server 224 includes a memory 226 to store blood pressure measurements, information regarding potential syncope episodes, indications of potential syncope episodes and/or current blood pressures, and/or the like, received from IMD 10 and/or external device 30, and processing circuitry 228, which may be configured to provide some or all of the functionality ascribed to processing circuitry 50 of IMD 10 and processing circuitry 200 of external device 30 herein. For example, processing circuitry 228 may determine the occurrence of the potential syncope episode from data sent by one or more IMDs 10 or external device 30. Processing circuitry 228 may determine the occurrence of the potential syncope episode in the manner described above with respect to processing circuitry 50 of IMD 10.

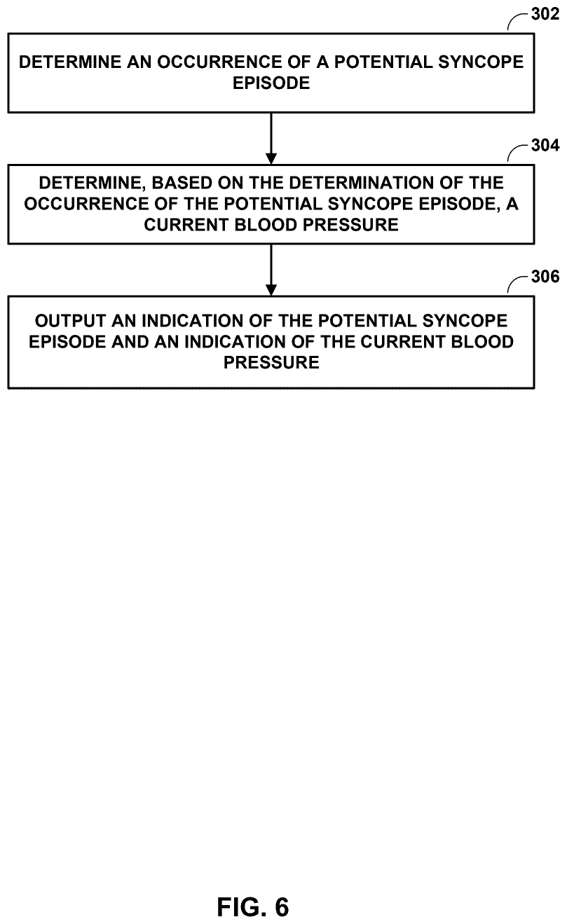

FIG. 6 is a flowchart illustrating example techniques for taking a blood pressure measurement for a potential syncope episode according to one or more aspects of this disclosure. While the techniques of FIG. 6 are described with respect to IMD 10, it should be understood that any of, or any combination of, devices described herein, or any other device capable of doing so, may perform the techniques of FIG. 6.

TNID 10 may determine an occurrence of a potential syncope episode (302). For example, to determine the occurrence of the potential syncope episode, TN/ID 10 may determine at least one of a) entering of patient input on an external device indicative of one or more patient symptoms of syncope, b) changes in a sensed neurological signal, or c) occurrence of a sensed sound of emesis. For example, IMD 10 may receive input of patient 14 indicative of patient 14 experiencing one or more symptoms of syncope and/or IMD 10 may determine changes in a neurological signal of patient 14 satisfy a threshold and/or IMD 10 may determine a sound of emesis. Based on one or more of such determinations, IMD 10 may determine an occurrence of a potential syncope episode.

IMD 10 may determine, based on the determination of the occurrence of the potential syncope episode, a current blood pressure (304). For example, IMD 10 may control a pressure sensor to take a current blood pressure measurement upon determination of the occurrence of the potential syncope episode. In some examples, the pressure sensor may include an optical sensor. In some examples, the pressure sensor may be part of a wearable blood pressure monitor. As stated above, determining blood pressure by an IMD may be a relatively high-power task, which may drain a battery relatively quickly if the IMD determines the blood pressure continually. As such, it may be desirable to determine the current blood pressure based on the determination of the occurrence of the potential syncope episode.

IMD 10 may output an indication of the potential syncope episode and an indication of the current blood pressure (306). For example, IMD 10 may control communication circuitry 54 to send an indication of the potential syncope episode and an indication of the current blood pressure to external device 30. For example, a clinician may use a measure of the current blood pressure, a measure of the baseline blood pressure, and/or other physiological parameter information associated with the potential syncope episode to verify that the potential syncope episode was a true syncope episode, to determine a potential cause of the potential syncope episode, to determine a potential treatment for the patient, and/or the like. In some examples, the clinician may use blood pressure measurements associated with a plurality of potential syncope episodes to verify that the potential syncope episode was a true syncope episode, to determine a potential cause of the potential syncope episode, to determine a potential treatment for the patient, and/or the like.

In some examples, to determine the occurrence of the potential syncope episode, IMD 10 may at least one of: determine a fall based on at least one accelerometer signal; determine changes in at least one of a heart rate or heart rhythm based on a sensed electrocardiogram signal; determine changes in respiration based on a sensed respiration signal; determine dizziness of the patient based on the at least one accelerometer signal; or determine that an accelerometer signal indicative of a change in posture meets a predetermined threshold. For example, to determine the occurrence of the potential syncope episode, ENID 10 may require more than one determination indicative of a potential syncope episode.

In some examples, IMD 10 may include at least one of sensing circuitry configured to sense at least one of the ECG signal, the respiration signal, or the neurological signal; an accelerometer configured to generate at least one signal indicative of a posture of the patient; or a microphone configured to sense a sound of the patient.

In some examples, IMD 10 may determine a time of day. IMD 10 may determine that the time of day is a predetermined time of day. IMD 10 may, based on the time of day being the predetermined time of day, determine a baseline blood pressure. IMD 10 may output an indication of the baseline blood pressure. In some examples, IMD 10 may compare the baseline blood pressure to the current blood pressure. In some examples, IMD 10 may determine, based on the comparison, at least one of contents of the indication or a priority of transmission of the indication.

In some examples, IMD 10 may determine an occurrence of a triggering event. IMD 10 may determine, based on the occurrence of the triggering event, a second blood pressure. IMD 10 may output an indication of the second blood pressure. In some examples, the triggering event includes at least one of the patient being in a predetermined posture or a rate of change of a posture of the patient satisfying a threshold.

In some examples, to determine the current blood pressure, IMD 10 may control at least one of an optical sensor or a wearable blood pressure monitor to generate a signal indicative of the current blood pressure. In some examples, IMD 10 may determine, based on the current blood pressure and the occurrence of the potential syncope episode, at least one of a potential reason for the occurrence of the potential syncope episode or recommended treatment. In some examples, IMD 10 may output an indication of the at least one of the potential reason or recommended treatment. In some examples, IMD 10 may receive symptom information of the patient. For example, IMD 10 may receive symptom information entered by patient 14 into UI 204 of external device 30 and/or symptom information from server 224 (e.g., from an electronic healthcare record of patient 14). This symptom information may be different than the patient input indicative of one or more patient symptoms of syncope and may include symptoms of patient 14 that are not necessarily symptoms of a syncope episode. For example, the symptom information might include headaches, chest pains, weariness, etc. In some examples, IMD 10 may determine the potential reason for the syncope episode further based on the symptom information.

In some examples, IMD 10 may determine, based on the determination of the syncope episode, a patient posture. ID 10 may output the indication of the patient posture.

This disclosure includes the following examples:

Example 1. A system comprising: one or more memories; and processing circuitry communicatively coupled to the one or more memories, the processing circuitry configured to: determine an occurrence of a potential syncope episode, wherein to determine the occurrence of the potential syncope episode the processing circuitry is configured to determine at least one of a) entering of patient input on an external device indicative of one or more patient symptoms of syncope, b) changes in a sensed neurological signal, or c) occurrence of a sensed sound of emesis; determine, based on the determination of the occurrence of the potential syncope episode, a current blood pressure; and output an indication of the potential syncope episode and an indication of the current blood pressure.

Example 2. The system of example 1, wherein to determine the occurrence of the potential syncope episode, the processing circuitry is further configured to at least one of: determine a fall based on at least one accelerometer signal; determine changes in at least one of a heart rate or heart rhythm based on a sensed electrocardiogram signal; determine changes in respiration based on a sensed respiration signal; determine dizziness of the patient based on the at least one accelerometer signal; or determine that an accelerometer signal indicative of a change in posture meets a predetermined threshold.

Example 3. The system of example 1 or example 2, further comprising at least one of: sensing circuitry configured to sense at least one of the electrocardiogram signal, the respiration signal, or the neurological signal; an accelerometer configured to generate at least one signal indicative of a posture of the patient; or a microphone configured to sense a sound of the patient.

Example 4. The system of any of examples 1-3, wherein the processing circuitry is further configured to: determine a time of day; determine that the time of day is a predetermined time of day; based on the time of day being the predetermined time of day, determine a baseline blood pressure; and output an indication of the baseline blood pressure.

Example 5. The system of example 4, wherein the processing circuitry is further configured to: compare the baseline blood pressure to the current blood pressure; and determine, based on the comparison, at least one of contents of the indication or a priority of transmission of the indication.

Example 6. The system of any of examples 1-5, wherein the processing circuitry is further configured to: determine an occurrence of a triggering event; determine, based on the occurrence of the triggering event, a second blood pressure; and output an indication of the second blood pressure.

Example 7. The system of example 6, wherein the triggering event comprises at least one of the patient being in a predetermined posture or a rate of change of a posture of the patient satisfying a threshold.

Example 8. The system of any of examples 1-7, wherein to determine the current blood pressure, the processing circuitry is configured to control at least one of an optical sensor or a wearable blood pressure monitor to generate a signal indicative of the current blood pressure.

Example 9. The system of any of examples 1-8, wherein the processing circuitry is further configured to: determine, based on the current blood pressure and the occurrence of the potential syncope episode, at least one of a potential reason for the occurrence of the potential syncope episode or recommended treatment; and output an indication of the at least one of the potential reason or recommended treatment.

Example 10. The system of example 9, wherein the processing circuitry is further configured to receive symptom information of the patient, and wherein determining the potential reason for the syncope episode is further based on the symptom information.

Example 11. The system of any of examples 1-10, wherein the processing circuitry is further configured to: determine, based on the determination of the syncope episode, a patient posture; and output an indication of the patient posture.

Example 12. A method comprising: determining, by processing circuitry, an occurrence of a potential syncope episode, wherein to determine the occurrence of the potential syncope episode the processing circuitry is configured to determine at least one of a) entering of patient input on an external device indicative of one or more patient symptoms of syncope, b) changes in a sensed neurological signal, or c) occurrence of a sensed sound of emesis; determining, by the processing circuitry and based on the determination of the occurrence of the potential syncope episode, a current blood pressure; and outputting, by the processing circuitry, an indication of the potential syncope episode and an indication of the current blood pressure.

Example 13. The method of example 12, wherein determining the occurrence of the potential syncope episode further comprises at least one of: determining, by the processing circuitry, a fall based on at least one accelerometer signal; determining, by the processing circuitry, changes in at least one of a heart rate or heart rhythm based on a sensed electrocardiogram signal; determining, by the processing circuitry, changes in respiration based on a sensed respiration signal; determining, by the processing circuitry, dizziness of the patient based on the at least one accelerometer signal; or determining, by the processing circuitry, that an accelerometer signal indicative of a change in posture meets a predetermined threshold.

Example 14. The method of example 12 or example 13, further comprising at least one of: sensing, by sensing circuitry, at least one of the electrocardiogram signal, the respiration signal, or the neurological signal; generating, by an accelerometer, at least one signal indicative of a posture of the patient; or sensing, by a microphone, a sound of the patient.

Example 15. The method of any of examples 12-14, wherein the method further comprises: determining, by the processing circuitry, a time of day; determining, by the processing circuitry, that the time of day is a predetermined time of day; determining, by the processing circuitry and based on the time of day being the predetermined time of day, a baseline blood pressure; and outputting, by the processing circuitry, an indication of the baseline blood pressure.

Example 16. The method of example 15, wherein the method further comprises: comparing, by the processing circuitry, the baseline blood pressure to the current blood pressure; and determining, by the processing circuitry and based on the comparison, at least one of contents of the indication or a priority of transmission of the indication.

Example 17. The method of any of examples 12-16, wherein the method further comprises: determining, by the processing circuitry, an occurrence of a triggering event; determining, by the processing circuitry and based on the occurrence of the triggering event, a second blood pressure; and outputting, by the processing circuitry, an indication of the second blood pressure.

Example 18. The method of example 17, wherein the triggering event comprises at least one of the patient being in a predetermined posture or a rate of change of a posture of the patient satisfying a threshold.

Example 19. The method of any of examples 12-18, wherein determining the current blood pressure comprises controlling at least one of an optical sensor or a wearable blood pressure monitor to generate a signal indicative of the current blood pressure.

Example 20. An implantable medical device comprising: one or more memories; and processing circuitry communicatively coupled to the one or more memories, the processing circuitry configured to: determine an occurrence of a potential syncope episode, wherein to determine the occurrence of the potential syncope episode the processing circuitry is configured to determine at least one of a) entering of patient input on an external device indicative of one or more patient symptoms of syncope, b) changes in a sensed neurological signal, or c) occurrence of a sensed sound of emesis; determine, based on the determination of the occurrence of the potential syncope episode, a current blood pressure; and output an indication of the potential syncope episode and an indication of the current blood pressure.

Various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, electrical stimulators, or other devices. The term “processor” or “processing circuitry” may generally refer to any of the foregoing logic circuitry, alone or in combination with other logic circuitry, or any other equivalent circuitry.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. Computer-readable media may include computer-readable storage media forming a tangible, non-transitory medium. Instructions may be executed by one or more processors, such as one or more DSPs, ASICs, FPGAs, general purpose microprocessors, or other equivalent integrated or discrete logic circuitry. Accordingly, the term “processor,” as used herein may refer to one or more of any of the foregoing structure or any other structure suitable for implementation of the techniques described herein.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

Various aspects of the disclosure have been described. These and other aspects are within the scope of the following claims.

What is claimed is:

1. A system comprising:
one or more memories; and
processing circuitry communicatively coupled to the one or more memories, the processing circuitry configured to:
determine an occurrence of a potential syncope episode, wherein to determine the occurrence of the potential syncope episode the processing circuitry is configured to determine at least one of a) entering of patient input on an external device indicative of one or more patient symptoms of syncope, b) changes in a sensed neurological signal, or c) occurrence of a sensed sound of emesis;
determine, based on the determination of the occurrence of the potential syncope episode, a current blood pressure; and
output an indication of the potential syncope episode and an indication of the current blood pressure.

2. The system of claim 1, wherein to determine the occurrence of the potential syncope episode, the processing circuitry is further configured to at least one of:
determine a fall based on at least one accelerometer signal;
determine changes in at least one of a heart rate or heart rhythm based on a sensed electrocardiogram signal;
determine changes in respiration based on a sensed respiration signal;
determine dizziness of the patient based on the at least one accelerometer signal; or
determine that an accelerometer signal indicative of a change in posture meets a predetermined threshold.

3. The system of claim 1, further comprising at least one of:
sensing circuitry configured to sense at least one of the electrocardiogram signal, the respiration signal, or the neurological signal;
an accelerometer configured to generate at least one signal indicative of a posture of the patient; or
a microphone configured to sense a sound of the patient.

4. The system of claim 1, wherein the processing circuitry is further configured to:
determine a time of day;
determine that the time of day is a predetermined time of day;
based on the time of day being the predetermined time of day, determine a baseline blood pressure; and
output an indication of the baseline blood pressure.

5. The system of claim 4, wherein the processing circuitry is further configured to:
compare the baseline blood pressure to the current blood pressure; and
determine, based on the comparison, at least one of contents of the indication or a priority of transmission of the indication.

6. The system of claim 1, wherein the processing circuitry is further configured to:
determine an occurrence of a triggering event;
determine, based on the occurrence of the triggering event, a second blood pressure; and
output an indication of the second blood pressure.

7. The system of claim 6, wherein the triggering event comprises at least one of the patient being in a predetermined posture or a rate of change of a posture of the patient satisfying a threshold.

8. The system of claim 1, wherein to determine the current blood pressure, the processing circuitry is configured to control at least one of an optical sensor or a wearable blood pressure monitor to generate a signal indicative of the current blood pressure.

9. The system of claim 1, wherein the processing circuitry is further configured to:
determine, based on the current blood pressure and the occurrence of the potential syncope episode, at least one of a potential reason for the occurrence of the potential syncope episode or recommended treatment; and
output an indication of the at least one of the potential reason or recommended treatment.

10. The system of claim 9, wherein the processing circuitry is further configured to receive symptom information of the patient, and wherein determining the potential reason for the syncope episode is further based on the symptom information.

11. The system of claim 1, wherein the processing circuitry is further configured to:
determine, based on the determination of the syncope episode, a patient posture; and
output an indication of the patient posture.

12. A method comprising:
determining, by processing circuitry, an occurrence of a potential syncope episode, wherein to determine the occurrence of the potential syncope episode the processing circuitry is configured to determine at least one of a) entering of patient input on an external device indicative of one or more patient symptoms of syncope, b) changes in a sensed neurological signal, or c) occurrence of a sensed sound of emesis;
determining, by the processing circuitry and based on the determination of the occurrence of the potential syncope episode, a current blood pressure; and
outputting, by the processing circuitry, an indication of the potential syncope episode and an indication of the current blood pressure.

13. The method of claim 12, wherein determining the occurrence of the potential syncope episode further comprises at least one of:
determining, by the processing circuitry, a fall based on at least one accelerometer signal;
determining, by the processing circuitry, changes in at least one of a heart rate or heart rhythm based on a sensed electrocardiogram signal;
determining, by the processing circuitry, changes in respiration based on a sensed respiration signal;
determining, by the processing circuitry, dizziness of the patient based on the at least one accelerometer signal; or
determining, by the processing circuitry, that an accelerometer signal indicative of a change in posture meets a predetermined threshold.

14. The method of claim 12, further comprising at least one of:
sensing, by sensing circuitry, at least one of the electrocardiogram signal, the respiration signal, or the neurological signal;
generating, by an accelerometer, at least one signal indicative of a posture of the patient; or
sensing, by a microphone, a sound of the patient.

15. The method of claim 12, wherein the method further comprises:
determining, by the processing circuitry, a time of day;
determining, by the processing circuitry, that the time of day is a predetermined time of day;
determining, by the processing circuitry and based on the time of day being the predetermined time of day, a baseline blood pressure; and
outputting, by the processing circuitry, an indication of the baseline blood pressure.

16. The method of claim 15, wherein the method further comprises:
comparing, by the processing circuitry, the baseline blood pressure to the current blood pressure; and
determining, by the processing circuitry and based on the comparison, at least one of contents of the indication or a priority of transmission of the indication.

17. The method of claim 12, wherein the method further comprises:
determining, by the processing circuitry, an occurrence of a triggering event;
determining, by the processing circuitry and based on the occurrence of the triggering event, a second blood pressure; and
outputting, by the processing circuitry, an indication of the second blood pressure.

18. The method of claim 17, wherein the triggering event comprises at least one of the patient being in a predetermined posture or a rate of change of a posture of the patient satisfying a threshold.

19. The method of claim 12, wherein determining the current blood pressure comprises controlling at least one of an optical sensor or a wearable blood pressure monitor to generate a signal indicative of the current blood pressure.

20. An implantable medical device comprising:
one or more memories; and
processing circuitry communicatively coupled to the one or more memories, the processing circuitry configured to:
determine an occurrence of a potential syncope episode, wherein to determine the occurrence of the potential syncope episode the processing circuitry is configured to determine at least one of a) entering of patient input on an external device indicative of one or more patient symptoms of syncope, b) changes in a sensed neurological signal, or c) occurrence of a sensed sound of emesis;
determine, based on the determination of the occurrence of the potential syncope episode, a current blood pressure; and
output an indication of the potential syncope episode and an indication of the current blood pressure.
